(19) United States
(12) Patent Application Publication
(10) Pub. No.: US 2016/0150997 A1
(43) Pub. Date: Jun. 2, 2016

(54) METHOD, DEVICE AND SYSTEM FOR DETERMINING AND UTILIZING ETCO2 TO PACO2 GRADIENT

(71) Applicant: ORIDION MEDICAL 1987 LTD., Jerusalem (IL)

(72) Inventors: Joshua Lewis Colman, Jerusalem (IL); Michal Ronen, Givat-Brenner (IL)

(21) Appl. No.: 14/556,161

(22) Filed: Nov. 30, 2014

(51) Int. Cl. A61B 5/083; A61B 5/1455; A61B 5/085; A61B 5/00; A61B 5/08

(52) U.S. Cl. CPC A61B 5/0836; A61B 5/7278; A61B 5/0816; A61B 5/085; A61B 5/7275; A61B 5/746; A61B 5/7282; A61B 5/742; A61B 5/7405; A61B 5/7455; A61B 5/14553

(57) ABSTRACT

Provided are medical monitoring systems, devices and methods of using the same, for estimating and providing information regarding EtCO₂, PaCO₂, gradient there between and trends thereof.

### TECHNICAL FIELD

The present disclosure generally relates to medical monitoring systems, devices and methods of using the same, for estimating and providing information regarding EtCO₂, PaCO₂, gradient there between and trends thereof.

### BACKGROUND

Medical monitoring devices are routinely used in various medical settings to obtain or measure medical parameters relating to a patient's medical condition. The parameters and measurements are used to determine the health condition of the subject and allow monitoring the condition over time. Carbon dioxide (CO₂) is produced as a by-product of metabolism and returned to the lungs via perfusion wherein it is them removed via alveolar ventilation. The difference (gradient) in the values between the arterial carbon dioxide partial pressure (PaCO₂) and the end tidal CO₂ partial pressure (etCO₂) is a result of the relationship between ventilation (airflow from the alveoli (alveoli CO₂ elimination) and perfusion (blood flow to the pulmonary capillaries and CO₂ diffusion into the alveoli). Determining the gradient requires obtaining a simultaneous arterial blood gas sample and an etCO₂ measurement.

### SUMMARY

According to some embodiments, there are provided monitoring devices, systems and methods, for an accurate, reliable and fast determination and/or estimation/evaluation of PaCO₂, EtCO₂, the gradient there between, and the trends they form and project. The devices, systems and methods disclosed herein can be used to more accurately determine the ventilation status of a subject and may further be used, in some embodiments, to prompt changes in ventilation care of the subject. In some embodiments, the devices, systems and methods disclosed herein can be used to more accurately determine the perfusion status of a subject and may further be used, in some embodiments, to prompt changes in care of the subject.

In some embodiments, the systems, devices and methods provided herein take use of measurements or related information obtained from one or more medical monitoring devices. In some embodiments, the medical monitoring devices may be selected from, but not limited to: capnograph, Breath Flow monitoring devices, INVOS (which can measure changes of cerebral perfusion), transcutaneous CO₂ measuring device, arterial blood gas sampling means, non-invasive devices for measuring PaCO₂ or estimation in changes thereof, and the like. In some embodiments, the measurements or related information obtained from the medical monitoring devices, may include various physiological parameters, such as, breath related parameters, blood related parameters, body temperature, and the like.

In some embodiments, the measurements or related information obtained from the medical monitoring devices are various physiological parameters, related to expired CO₂ and blood gasses, including, for example, EtCO₂ and PaCO₂.

Advantageously, the systems, devices and methods disclosed herein, allow accurate and safe measurements and/or estimations/evaluations of PaCO₂ and EtCO₂, the gradient there between and the trends they create and project, so as to provide vital information regarding the ventilation and/or perfusion status of the subject and to further use the information obtained and prompt changes in ventilation care of the subject. The systems, devices and methods disclosed herein are advantageous over method currently employed. Measurements of PaCO₂ alone, for example by transcutaneous measurements or arterial blood gas sampling (ABG) are not continuous and can not directly indicate a breath related disorder. Further, the use of such devices may be inherently limited. For example, transcutaneous measurements of CO₂ in the blood require locally heating the sensor (electrode) which is in contact with the skin, a step which limits the use of the sensor for an extended period of time, and requires moving the sensor between different skin locations. Likewise, the use of arterial blood gas sampling is limited since it is not continuous and in some subjects, such as neonates it is not efficient. Hence, the systems, devices and methods disclosed herein advantageously provide for a safe and accurate measurement of PaCO₂ and EtCO₂, determination of the gradient between them, and the trends they create, as well as to further provide vital information derived from these measurements, in particular with respect to the ventilation and/or respiration status of the subject.

According to some embodiments, there is provided a system for determining, evaluating and/or estimating the gradient between arterial carbon dioxide partial pressure (PaCO₂) and end tidal carbon dioxide partial pressure (EtCO₂) of a subject, the system comprising:
a) A capnograph configured to provide continuous measurements of EtCO₂ and one or more additional breath related parameters of the subject; and
b) A processing unit configured to:
   a) provide estimation of PaCO₂ based on the measurements provided by the capnograph; and
   b) estimating the gradient between arterial carbon dioxide partial pressure (PaCO₂) and end tidal carbon dioxide partial pressure (EtCO₂), based on the measurements of EtCO₂ and the estimation of the PaCO₂.

In some embodiments, the additional breath related parameters may be selected from, but not limited to: breath flow, breath flow waveform, respiration rate (RR), respiration effort, dead space, CO₂ waveform, CO₂ volumetric waveform, data derived therefrom, and the like or combinations thereof. In some embodiments, the data derived from the CO₂ volumetric waveform may include for example, but not limited to slope of stage III of said waveform.

In some embodiments, the processing unit may be configured to provide volumetric capnography, based on the measurements of the capnograph.

According to additional embodiments, there is provided a system for determining the gradient between arterial carbon dioxide partial pressure (PaCO₂) and end tidal carbon dioxide partial pressure (EtCO₂) of a subject, the system comprising:

a) A first monitoring device configured to provide continuous measurements of EtCO₂;

b) A second monitoring device configured to provide measurements related to breath flow; and

c) A processing unit configured to obtain measurements from the first and second monitoring devices and to provide estimation of the gradient between the PaCO₂ and the EtCO₂ based on said measurements.

In some embodiments, the system may further include a third monitoring device configured to provide measurements related to PaCO₂; wherein the third monitoring device is prompted/controlled/triggered/operated by the first monitoring device.

According to some embodiments, the first monitoring device is a capnograph. In some embodiments, the capnography measurements may be volumetric capnography.

According to some embodiments, the second monitoring device may be configured to provide one or more additional breath related parameters. In some embodiments, the breath related parameters may be selected from, but not limited to: respiration rate (RR), respiration effort, breath flow, CO₂ waveform, physiological dead space, data derived therefrom, and the like or combinations thereof. In some embodiments, the second monitoring device may be selected from a monitoring device capable of indirectly measure perfusion, such as, for example, but not limited to: plesysmograph of the SpO2, Cerebral Oxymeter, and the like.

In some embodiments, the third monitoring device may be selected from any monitoring device, such as, perfusion monitoring device capable of measuring PaCO₂, such as, transcutaneous CO₂ measuring device, non-invasive PaCO₂ monitoring device, means for obtaining arterial blood gas samples, and the like.

According to some embodiments, the third device may be prompted/activated/triggered/operated by the first monitoring device intermittently. In some embodiments, the prompt/activation of the third measuring device may be initiated if one or more of the values determined by the processing unit (for example, EtCO₂, PaCO₂, gradient between PaCO₂, and EtCO₂, changes to the gradient), deviate from a predetermined threshold.

According to some embodiments, the processing unit is further configured to determine a change of the gradient between the PaCO₂ and the EtCO₂, over time (that is, determine the trend of the gradient).

According to some embodiments, the system may further include an alert unit configured to issue an alert if one or more of the values determined by the processing unit (for example, EtCO₂, PaCO₂, gradient between PaCO₂, and EtCO₂, changes to the gradient), deviate from a predetermined threshold. In some embodiments, the alert may be selected from tactile, visual and audible.

In some embodiments, the system may further include a user interface configured to control operating parameters of the system.

According to some embodiments, there is provided a method for determining the gradient between arterial carbon dioxide partial pressure (PaCO₂) and end tidal carbon dioxide partial pressure (EtCO₂) of a subject, the method comprising one or more of the steps of:
a. obtaining continuous measurements of EtCO₂ and measurements of one or more additional breath related parameters;
b. estimating PaCO₂ based on the continuous measurements of EtCO₂ and the one or more additional breath related parameters;
c. determining the gradient between the estimated PaCO₂ and measured EtCO₂.

In some embodiments, the method may further include a step of obtaining a second measurement related to PaCO₂; wherein the second measurement is obtained if the measurements of step a) and/or step b) deviate from a predetermined threshold.

In some embodiments, the one or more additional breath related parameters may be selected from, but not limited to: respiration rate (RR), respiration effort, breath flow, breath flow waveform, volumetric waveform, CO₂ waveform, data derived therefrom, and the like or combinations thereof. In some embodiments the EtCO2 measurements may be obtained by a capnograph.

In some embodiments, the second measurement may be obtained by a transcutaneous blood gas sensor, non-invasive PaCO₂ monitoring device, arterial blood gas sampling line, plesysmograph of the SpO₂, Cerebral Oxymeter, and the like, or combinations thereof.

In some embodiments, the method may further include estimating/determining the trend of the gradient.

In some embodiments, the method may further include issuing as alert if one or more of EtCO₂ and/or the parameters related thereto and/or the PaCO₂ and/or the gradient and/or the trend of the gradient deviate from a predetermined threshold.

In some embodiments, the subject being monitored is an intubated patient. In some embodiments, the subject being monitored is a non-intubated patient. In some embodiments, the subject being monitored is sedated. In some embodiments, the systems, devices and methods disclosed herein may be used in various health care settings, such as, for example, but not limited to: Intensive care (ICU), pain management, operation room, and the like. The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

### BRIEF DESCRIPTION OF THE FIGURES

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure. For the sake of clarity, some objects depicted in the figures are not to scale.

FIG. 1—a schematic block diagram of a system for estimating/determining a gradient between PaCO₂ and EtCO₂, according to some embodiments;

FIG. 2—a schematic block diagram of steps in a method for estimating/determining a gradient between PaCO₂ and EtCO₂, according to some embodiments; and

FIG. 3—an exemplary block diagram of method for estimating/determining a gradient between PaCO₂ and EtCO₂, according to some embodiments.

### DETAILED DESCRIPTION

In the following description, various aspects of the disclosure will be described. For the purpose of explanation, specific configurations and details are set forth in order to provide a thorough understanding of the disclosure. However, it will also be apparent to one skilled in the art that the disclosure may be practiced without specific details being presented herein. Furthermore, well-known features may be omitted or simplified in order not to obscure the disclosure.

As referred to herein, the terms “user”, “medical user”, “health care provider” and “health care professional” may interchangeably be used. The terms may include any health care provider who may treat and/or attend to a patient. A user may include, for example, a nurse, respiratory therapist, physician, anesthesiologist, and the like. In some cases, a user may also include a patient.

As referred to herein, the terms “monitoring device” and “medical device” may interchangeably be used. Exemplary monitoring devices include such devices as, but not limited to: Capnograph, pulse oximeter, perfusion monitoring device, arterial blood sampling means, transcutaneous CO₂ measuring device, non-invasive PaCO₂ monitoring device, breath flow measuring device, and the like, or combinations thereof.

As referred to herein, the term “physiological parameter” is directed to a health related parameter of the subject. The health related parameter may be directly and/or indirectly measured, detected and/or derived from a measurement of a medical monitoring device, for example, via an appropriate sensor. In some embodiments, the health related parameter may include such parameters as, but not limited to: breath related parameters, ventilation related parameters and/or blood gases related parameters, such as, for example, CO₂ related parameters, EtCO₂, PaCO₂, O₂ related parameters, breath rate, breath cycle, respiration rate, breath flow, V_{D}/V_{T}, and the like.

As referred to herein, the terms “patient” and subject” may interchangeably be used and may relate to a subject being monitored by any monitoring device for any physical-condition related parameter and/or health related parameter. In some embodiments, the subject is capable of responding to instructions provided by a communicating device or system.

As referred to herein, the term “waveform” is directed to a recurring graphic shape which may be realized by measuring a physiological parameter of a subject over time or over other parameters (such as, volume), such as, for example, concentration of CO₂ in breath, breath flow, rate of breath, electrocardiogram (ECG), plethysmograph, and the like. In some embodiments, a waveform is a medically, time resolved waveform. A waveform may have various characteristic parameters/features/factors that may be derived from the shape, dimension, rate or frequency, reoccurrences, and the like, and combinations thereof.

As referred to herein, the terms ordinary, normal, typical, standard and common may interchangeably be used.

As referred to herein, the term “EtCO₂” relates to End tidal CO₂ partial pressure. The CO₂ is exhaled out of the body and the concentration of the exhaled CO₂, also known as end tidal CO₂ (EtCO₂) is an approximate estimation of the alveolar CO₂ pressure and thus of the arterial levels of CO₂. The values of EtCO₂ may be measured in units of pressure, such as, for example, mmHg.

As referred to herein, the term “PaCO₂” relates to arterial carbon dioxide partial pressure (PaCO₂). PaCO₂ is indicative of the partial CO₂ pressure in the alveoli. The values of PaCO₂ may be measured in units of pressure, such as, for example, mmHg.

As referred to herein the terms “gradient” and “gradient between PaCO₂ and EtCO₂” may interchangeably be used. The terms are directed to the difference between the PaCO₂ and the EtCO₂ values. The gradient results from the relationship between ventilation (airflow to the alveoli) and perfusion (blood flow to the pulmonary capillaries. For example, in normal, healthy lungs, there is a good match of alveolar ventilation and perfusion to pulmonary capillaries, resulting in an EtCO₂ value that closely correlates with or matches the PaCO₂ value.

As referred to herein, the term “trend” is directed to a change over time of a measured or determined value. For example, in some embodiments, trend of the gradient is directed to include change over time of the gradient between PaCO₂ and EtCO₂.

As referred to herein, the term “breath cycle” includes the stages of exhalation and inhalation. The breath cycle may be derived from a CO₂ waveform which depicts the change in expired CO₂ Volume over time, (EtCO₂). During a breath cycle, the levels of CO₂ initially increase as a result of CO₂ release from the airways, from what is known as the “dead space”, which is the space in which no gas exchange takes place. Then, the CO₂ rapidly reaches a plateau at high levels of CO₂, which corresponds to the release of CO₂ from the lungs, in the exhalation phase. A rapid decline in exhaled CO₂ proceeds the inhalation phase, characterized by absence/minute levels of CO₂.

As referred to herein, the term “Respiration Rate” (RR) may be defined as the number of breaths taken in a minute, and it may change under various physiological and medical conditions.

In some embodiments, the terms “calculated”, “determined” and “computed” may interchangeably be used.

The term “condition” is directed to the physiological (health) condition of the subject, and/or changes to the condition over time.

As referred to herein, the terms “dead space” and “physiological dead space” may interchangeably be used and are directed to the volume of air which is inhaled that does not take part in the gas exchange, either because it (1) remains in the conducting airways, or (2) reaches alveoli that are not perfused or poorly perfused. Physiological dead space ventilation is the sum of anatomical dead space from the conducting airways and alveolar dead space. The ratio between the dead space to the tidal volume is indicated herein by the formula: V_{D}/V_{T}.

Capnography is a non-invasive monitoring method used to continuously measure CO₂ concentration in exhaled breath. The CO₂, which is a constant metabolism product of the cells, is exhaled out of the body, and the concentration of the exhaled CO₂, also known as end tidal CO₂ (EtCO₂), is an approximate estimation of the arterial levels of CO₂. Capnograph (or capnometer) is a medical monitoring device that may be used for measuring the carbon dioxide (CO₂) content in inspired and expired air of a subject and is indicative of ventilation of the respiratory system. It is a non-invasive device that measures the concentrations of respired gases. Capnography may include time-based and/or volumetric capnography. In some embodiments, for time-based capnography the concentration of carbon dioxide is displayed in time format (i.e., change in exhaled CO2 over time). For volumetric capnography, exhaled PCO₂ is plotted/displayed versus exhaled volume.

Transcutaneous carbon dioxide monitoring device is a non-invasive monitoring tool that allows the determination (among other values) of PaCO2. Transcutaneous measurements of PaCO₂ are based on the principal that a heating element in a measuring electrode (sensor) elevates the temperature of the underlying tissues below the skin. This increase capillary blood flow and the partial pressure of CO2, making the skin permeable to gas diffusion, which allows the measurement of the gas and determination of PaCO₂, while taking into account the elevated temperature and other deviations from the real arterial values of CO₂. The accuracy and sensitivity of the measurements may be dependent on the extent of the elevated temperature of the sensor. Additional analysis of blood gases may be performed by standard blood gas analysis (ABG), in which blood sample is taken from the subject and analyzed for various blood gas related parameters, such as, pH of the blood, the partial pressure of carbon dioxide and oxygen, bicarbonate level, in addition to other parameters, such as, concentration of lactate, hemoglobin, electrolytes, oxyhemoglobin, carboxyhemoglobin methemoglobin, and the like.

In respiratory physiology, ventilation is directed to the air that reaches the alveoli and perfusion is directed to the blood that reaches the alveoli

According to some embodiments, there are provided systems and methods for estimating, evaluating and/or determining the gradient between arterial carbon dioxide partial pressure (PaCO₂) and end tidal carbon dioxide partial pressure (EtCO₂), of a subject. The systems and methods further allow for estimating/determining various related parameters, such as the trend of the gradient, efficiency of ventilation, and the like. The systems and methods provided herein allow for a more accurate and reliable continuous determination and evaluation of PaCO₂, the gradient and changes thereto, to ultimately provide a better indication as to the status of the subject. In some embodiments, the continuous evaluation of the gradient provides for a safer, more reliable and accurate evaluation of the gradient. In some embodiments, in addition to the continuous evaluation of the gradient, additional, intermittent, direct determination of the PaCO₂ and the gradient) may be performed, wherein the additional determination of the gradient is initiated when the estimated gradient (or PaCO₂) deviates from various thresholds. By this, a far more accurate and reliable determination of the gradient may be obtained (for example, when using Transcutaneous measurements only periodically, for direct determination of PaCO₂, higher sensor temperature may be used, which results in enhanced sensitivity and more accurate results).

According to some embodiments, capnography measurements may provide indication as to the ventilation of the respiration system, but it does not provide a direct measurement of the perfusion (i.e. blood reaching the alveoli). Hence, it may not provide a direct or an accurate assessment of CO₂ in the blood. The importance of CO₂ concentration in the blood is critical, in particular due to the body's use of CO₂ as a buffering agent of the blood. Accordingly, PaCO₂ measurements should also be obtained in a timely manner, so as gain information regarding the perfusion status of the subject and to thus have a more accurate indication as to the overall condition of the subject.

In some embodiments, the systems, devices and methods provided herein take use of measurements of breath related parameters/or information/data derived therefrom, obtained from a capnograph. In some embodiments, a capnograph is used to provide continuous measurements of breath related parameters (such as, EtCO₂), which provide the “baseline” for estimating, calculating and/or determining the gradient. In some embodiments, the systems, devices and methods provided herein take use of additional measurements of physiological parameters and/or information/data derived therefrom, obtained from one or more medical monitoring devices that may interact with each other. In some embodiments, the medical monitoring devices may be selected from, but not limited to: arterial blood gas sampling line, Transcutaneous CO₂ measurements, non-invasive device for measuring PaCO₂, and the like. In some embodiments, the physiological parameters, or data derived therefrom, are such parameters as, for example, but not limited to: breath related parameters (as determined, for example, based on expired CO₂ (measurements of CO₂ in exhaled breath), breath flow, dead space, blood related parameters (such as, blood gases, PaCO₂, and the like), body temperature, and the like, or combinations thereof.

According to some embodiments, there is provided a system for determining, evaluating and/or estimating the gradient between arterial carbon dioxide partial pressure (PaCO₂) and end tidal carbon dioxide partial pressure (EtCO₂) of a subject, the system comprising:
a) A capnograph configured to provide continuous measurements of EtCO₂ and one or more additional breath related parameters of the subject; and
b) A processing unit configured to:
   a) provide estimation of PaCO₂ based on the measurements provided by the capnograph; and
   b) determine the gradient between arterial carbon dioxide partial pressure (PaCO₂) and end tidal carbon dioxide partial pressure (EtCO₂), based on the measurements of EtCO₂ and the estimation of the PaCO₂.

In some embodiments, the additional breath related parameters may be selected from, but not limited to: breath flow, breath flow waveform, respiration rate (RR), respiration effort, dead space, CO₂ waveform, CO₂ volumetric waveform, data derived therefrom, and the like or combinations thereof. In some embodiments, the data derived from the CO₂ volumetric waveform may include for example, but not limited to slope of stage III of said waveform.

In some embodiments, the processing unit may be configured to provide volumetric capnography, based on the measurements of the capnograph.

According to additional embodiments, there is provided a system for determining/estimating the gradient between arterial carbon dioxide partial pressure (PaCO₂) and end tidal carbon dioxide partial pressure (EtCO₂) of a subject, the system comprising:

a) A first monitoring device configured to provide continuous measurements of EtCO₂;

b) A second monitoring device configured to provide measurements related to breath flow; and

c) A processing unit configured to obtain measurements from the first and second monitoring devices and to determine the gradient between the PaCO₂ and the EtCO₂ based on said measurements.

In some embodiments, the system may further include a third monitoring device configured to provide measurements related to PaCO₂; wherein the third monitoring device is prompted/controlled/triggered/operated by the first monitoring device.

According to some embodiments, the first monitoring device is a capnograph. In some embodiments, the capnography measurements may be volumetric capnography.

According to some embodiments, the second monitoring device may be configured to provide one or more additional breath related parameters. In some embodiments, the breath related parameters may be selected from, but not limited to: respiration rate (RR), respiration effort, breath flow, CO₂ waveform, physiological dead space, data derived therefrom, and the like or combinations thereof:

In some embodiments, the third monitoring device may be selected from any perfusion monitoring device, such as, transcutaneous CO₂ measuring device, non-invasive PaCO₂ monitoring device, and means for obtaining arterial blood gas samples.

According to some embodiments, the third device may be prompted/activated/triggered/operated by the first monitoring device intermittently. In some embodiments, the prompt/activation of the third measuring device may be initiated if one or more of the values determined by the processing unit (for example, EtCO₂, PaCO₂, gradient between PaCO₂, and EtCO₂, changes to the gradient), deviate from a predetermined threshold.

According to some embodiments, there is provided a system for determining, evaluating and/or estimating the gradient between arterial carbon dioxide partial pressure (PaCO₂) and end tidal carbon dioxide partial pressure (EtCO₂) of a subject, the system comprising:

a) A first monitoring device configured to provide continuous measurements of EtCO₂;

b) A second monitoring device configured to provide measurements related to PaCO₂; wherein the second monitoring device is controlled/triggered/operated by the first monitoring device; and

c) A processing unit configured to obtain measurements from the first and second monitoring devices and to determine the gradient between the PaCO₂ and the EtCO₂.

According to some embodiments, the first monitoring device is a capnograph. In some embodiments, the second monitoring device may be selected from transcutaneous CO₂ measuring device, non-invasive CO₂ measuring device, and means for Arterial blood gas sampling.

According to some embodiments, the first monitoring device is configured to provide one or more additional breath related parameters. In some embodiments, the breath related parameters may be selected from, but not limited to: respiration rate (RR), respiration effort, dead space and parameters related thereto, breath flow, airflow, CO₂ waveform, data derived therefrom, and the like or combinations thereof.

According to some embodiments, the second device is activated/triggered/operated by the first monitoring device intermittently. In some embodiments, the activation of the second measuring device is initiated in response to deviation of one or more measurements obtained of the first monitoring device as compared to a predetermined threshold (as further detailed below).

According to some embodiments, the processing unit is further configured to determine a change of the gradient between the PaCO₂ and the EtCO₂, over time (that is, determine the trend of the gradient).

According to some embodiments, the system may further include an alert unit configured to issue an alert if one or more of the values measured by the monitoring devices and/or determined by the processing unit deviate from a predetermined threshold. In some embodiments, the alert may be selected from tactile alert, visual alert and/or audible alert.

According to some embodiments, the values/parameters the may deviate from the predetermined threshold may include such values as, but not limited to: EtCO₂, PaCO₂, respiration rate, respiration effort, breath flow, breath flow waveform, slope of stage III of breath flow waveform, dead space and parameters related thereto, EtCO₂ waveform, gradient between PaCO₂ and EtCO₂, trend of the gradient, changes of any one of the values, and the like, or combinations thereof. In some exemplary embodiments, values/parameters indicative of change in gradient may further include changes in EtCO₂ while CO₂ waveform remains normal (i.e., good lung mechanics, but poor perfusion), changes in blood pressure than may indicate possible lower perfusion, and the like, or combinations thereof.

In some embodiments, the measured parameters may be further manipulated and/or processed to generate data related to the measurements, prior to or concomitantly while being manipulated/used by the processing unit. For example, in some embodiments, various CO₂ related parameters may be derived from the measurement of expired CO₂, such as, for example but not limited to: CO₂ waveform and parameters related thereto, (such as, for example, but not limited to: changes in EtCO₂, a slope of the increase in the CO₂ concentration, a change in a slope of the increase in the CO₂ concentration, time to rise to a predetermined percentage of a maximum value of CO₂ concentration, a change in time to rise to a predetermined percentage of a maximum value of CO₂ concentration, an angle of rise to a predetermined percentage of a maximum value of CO₂ concentration, a change in an angle of rise to a predetermined percentage of a maximum value of CO₂ concentration, breath to breath correlation, a change in breath to breath correlation, a CO₂ duty cycle, a change in CO₂ duty cycle, minute ventilation, a change in minute ventilation,), and the like, or any combination thereof), volumetric capnography waveform and parameters related thereto (for example, slope of stage III of the volumetric waveform) respiration rate, breath cycle.

In some embodiments, the measurements related to the expired CO₂ and/or the data related thereto as well as changes or deviations in any of these values from a predetermined threshold may be used to determine that blood gases measurements is due. Such indications include, for example, but not limited to: increasing or decreasing levels of EtCO₂, increasing or decreasing respiration rate compared to a predetermined threshold, indications for low perfusion (low EtCO₂ with normal waveform shape), changes in CO₂ waveform, changes in volumetric waveform (expired CO₂ over expired volume), changes in slope of the stage III of the volumetric waveform, and the like, or combinations thereof.

According to some embodiments, the measured physiological parameters may be determined or calculated over a period of time, in order to effectively determine the condition of the subject over time. The period of time may be predetermined.

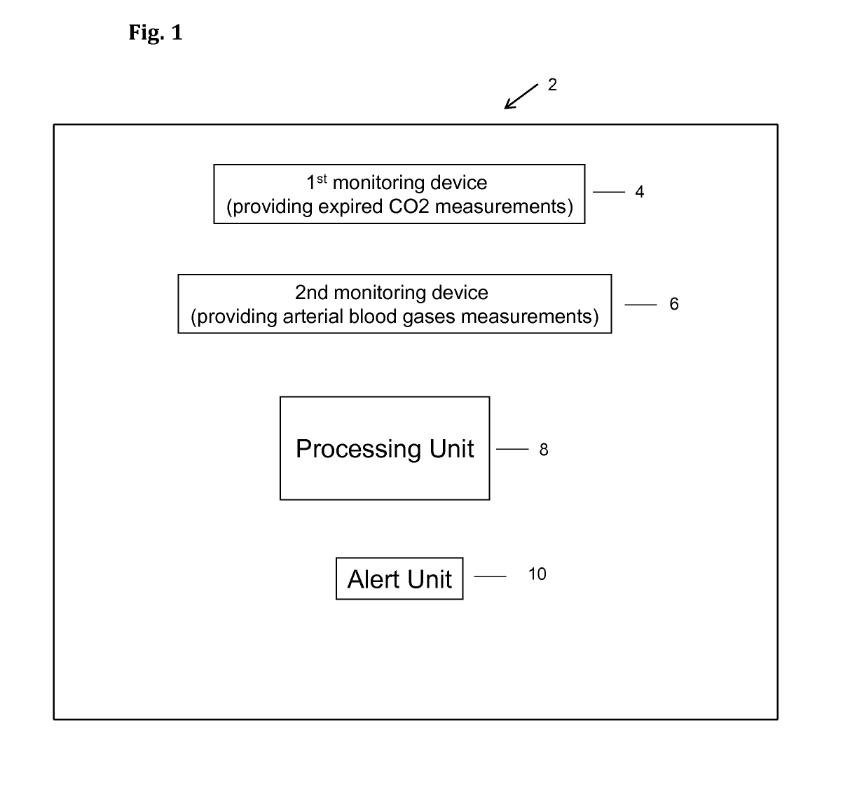

Reference is now made to FIG. 1, which is a schematic block diagram of a system which includes a first monitoring device and a second monitoring device, according to some embodiments. As shown in FIG. 1, the system (2) may include a first monitoring device, (4), capable of obtaining measurements of expired CO₂ and optionally additional related parameters. The system may further include a second monitoring device (6) capable of obtaining measurements of blood gases, including, for example, PaCO₂. The second device may be activated by the first monitoring device if/when one or more of the parameters obtained by the first monitoring device deviate from a predetermined threshold. The system may further include a processing unit (8) capable of obtaining measurements from the first and second monitoring devices and to determine the values related to the measurements, such as, for example, the gradient between the PaCO₂ and the EtCO₂, the trend of the gradient, and the like. The system may further include an alert unit (10), capable of issuing an alert if the values measured by the monitoring devices and/or determined by the processing unit deviate from a predetermined threshold. The system may further include a user interface that may include any type of interface allowing a user to control various operating parameters of the system. The connection between the medical monitoring device(s) and/or the processing unit and/or any other optional units may include any type of communication route, such as, for example, use of wires, cables, wireless, and the like.

According to some embodiments, the systems disclosed herein may relay information of the ventilation or respiration and/or perfusion and/or metabolism status of the subject. Based on the measured parameters or changes thereto, the systems may be used to prompt a health care provider, to inform of improvement or deterioration in the condition of the subject. In some embodiments, the systems may further be configured to automatically evoke changes in ventilation of the subject, in order to maintain the measured/determined parameters (such as, for example, gradient, PaCO₂ values) within defined levels (values).

According to some embodiments, the first monitoring device (for example, capnograph), may be used for turning on or triggering operation of a second monitoring device (for example, transcutaneous CO₂ measuring device). For example, the transcutaneous sensor may be activated (heating-up) only when defined events, indications and thresholds from the capnograph are recognized, (for example, increasing or decreasing levels of EtCO₂, increasing or decreasing Respiration rate above given threshold, indications for low perfusion (low EtCO₂ with normal waveform shape), and the like. In this way, the transcutaneous measurement will be used intermittently and only when there are clear indications for its necessity. In some embodiments, the transcutaneous sensor activation may be also triggered manually by a health care provider, based on the parameters obtained by the first monitoring device. In this manner, the heating process of the sensor (and consequently, the subject skin), would be reduced to a minimum time period and duty cycle, reducing considerably the issues with burns, the need for moving the sensor to different locations on the skin and frequent calibrations.

According to some embodiments, the first monitoring device (for example, a capnograph), may be used for triggering operation of the second monitoring device (for example, transcutaneous CO₂ measuring device). For example, the transcutaneous sensor may be operating at low levels (for example, at low temperature) and be triggered to elevate measuring temperature (heating-up) only when defined events, indications and thresholds from the capnograph are recognized, (for example, increasing or decreasing levels of EtCO₂, increasing or decreasing Respiration rate above given threshold, indications for low perfusion (low EtCO₂ with normal waveform shape), changes in V_{D}/V_{T}, and the like. In this way, the transcutaneous measurement will be used intermittently and only when there are clear indications for its necessity. In some embodiments, the transcutaneous sensor activation may be also triggered manually by a health care provider, based on the parameters obtained by the first monitoring device. In this manner, the heating process of the sensor (and consequently, the subject skin), would be reduced to a minimum time period and duty cycle, reducing considerably the issues with burns, the need for moving the sensor to different locations on the skin and frequent calibrations.

According to some embodiments, the systems and methods disclosed herein may provide for vital information regarding the status of the subject. For example, the systems and methods allow the projection and creation of trends of gradient, which itself provides vital information regarding efficiency of ventilation, ventilation quotient, perfusion, metabolism, well of being, and the like, in addition to providing a far greater overall perspective of the patient condition, where mismatches or deviations from predetermined threshold may be attributed to various medical conditions, such as pneumonia (liquid in lungs), low perfusion, Pulmonary Embolism, blocking of airway and the like.

According to some embodiments, there is provided a method for determining/estimating the gradient between arterial carbon dioxide partial pressure (PaCO₂) and end tidal carbon dioxide partial pressure (EtCO₂) of a subject, the method comprising one or more of the steps of:
a. obtaining continuous measurements of EtCO₂ and measurements of one or more additional breath related parameters;
b. estimating PaCO₂ based on the continuous measurements of EtCO₂ and the one or more additional breath related parameters;
c. estimating the gradient between the estimated PaCO₂ and measured EtCO₂.

According to some embodiments, the parameters related to EtCO₂ may include such parameters as, but not limited to: respiration rate (RR), respiration effort, breath flow, volumetric waveform and parameters related thereto (such as, for example, slopes of the waveform, slope of stage III of the waveform, and the like), CO₂ waveform and parameters related thereto (such as, for example, but not limited to: EtCO₂, changes in EtCO₂, a slope of the increase in the CO₂ concentration, a change in a slope of the increase in the CO₂ concentration, time to rise to a predetermined percentage of a maximum value of CO₂ concentration, a change in time to rise to a predetermined percentage of a maximum value of CO₂ concentration, an angle of rise to a predetermined percentage of a maximum value of CO₂ concentration, a change in an angle of rise to a predetermined percentage of a maximum value of CO₂ concentration, breath to breath correlation, a change in breath to breath correlation, a CO₂ duty cycle, a change in CO₂ duty cycle, minute ventilation, a change in minute ventilation or any combination thereof), breath cycle, and the like, or any combination thereof.

In some embodiments, the method may further include a step of obtaining a second measurement related to PaCO₂; wherein the second measurement is obtained if the measurements of step a) and/or step b) deviate from a predetermined threshold.

According to additional embodiments, there is provided a method for determining/estimating the gradient between arterial carbon dioxide partial pressure (PaCO₂) and end tidal carbon dioxide partial pressure (EtCO₂) of a subject, the method comprising one or more of the steps of:
a. obtaining continuous measurements of EtCO₂ and parameters related thereto;
b. obtaining a second measurement related to PaCO₂ wherein the second measurement is obtained if the measurements of step a) deviate from a predetermined threshold; and
c. determining the gradient between the PaCO₂ and EtCO₂ based on the measurements obtained.

According to some embodiments, the parameters related to EtCO₂ may include such parameters as, but not limited to: respiration rate (RR), respiration effort, breath flow, volumetric waveform and parameters related thereto (such as, for example, slopes of the waveform, slope of stage III of the waveform, and the like), CO₂ waveform and parameters related thereto (such as, for example, but not limited to: EtCO₂, changes in EtCO₂, a slope of the increase in the CO₂ concentration, a change in a slope of the increase in the CO₂ concentration, time to rise to a predetermined percentage of a maximum value of CO₂ concentration, a change in time to rise to a predetermined percentage of a maximum value of CO₂ concentration, an angle of rise to a predetermined percentage of a maximum value of CO₂ concentration, a change in an angle of rise to a predetermined percentage of a maximum value of CO₂ concentration, breath to breath correlation, a change in breath to breath correlation, a CO₂ duty cycle, a change in CO₂ duty cycle, minute ventilation, a change in minute ventilation or any combination thereof), breath cycle, and the like, or any combination thereof.

According to some embodiments, the method may further include a step of determining/estimating the trend of the gradient. In some embodiments, the method may further include a step of issuing an alert if the measured parameters and/or gradient and/or the trend of the gradient deviate from a predetermined threshold.

In some embodiments, the method may be used to more accurately determine the ventilation and/or perfusion status of the subject, to more precisely identify the underlying condition.

According to some embodiments, there is provided a method used in a system for determining, estimating and/or evaluating the gradient between arterial carbon dioxide partial pressure (PaCO₂) and end tidal carbon dioxide partial pressure (EtCO₂) of a subject, the system comprising: A capnograph configured to provide continuous measurements of EtCO₂ and one or more additional breath related parameters; and b) A processing unit configured to: a) provide estimation of PaCO₂ based on the measurements provided by the capnograph; and b) determine the gradient between arterial carbon dioxide partial pressure (PaCO₂) and end tidal carbon dioxide partial pressure (EtCO₂), based on the measurements of EtCO₂ and the estimation of the PaCO₂.

According to some embodiments, there is provided a method used in a system for determining, evaluating and/or estimating the gradient between arterial carbon dioxide partial pressure (PaCO₂) and end tidal carbon dioxide partial pressure (EtCO₂) of a subject, the system comprising: A first monitoring device configured to provide continuous measurements of EtCO₂; b) A second monitoring device configured to provide measurements related to PaCO₂; wherein the second monitoring device is controlled/triggered/operated by the first monitoring device; and c) A processing unit configured to obtain measurements from the first and second monitoring devices and to determine the gradient between the PaCO₂ and the EtCO₂.

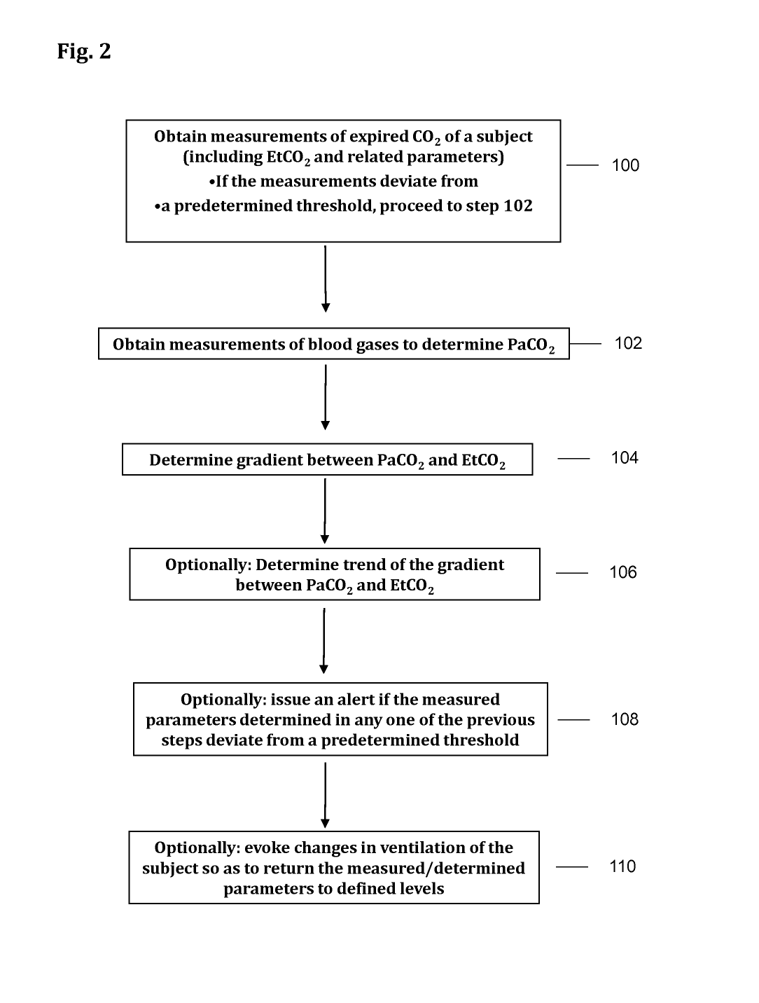

Reference is now made to FIG. 2, which is a schematic block diagram of steps in a method for determining, estimating and/or evaluating the gradient between arterial carbon dioxide partial pressure (PaCO₂) and end tidal carbon dioxide partial pressure (EtCO₂), according to some embodiments. As shown in FIG. 2, measurements of expired CO₂ of a subject are obtained (step 100). The measurements may be obtained continuously. Optionally, additional parameters related to the measurements of expired CO₂ are obtained/determined (for example, breath flow, volumetric waveform, and the like). Next, if the obtained measurements of expired CO₂ and/or the parameters related thereto deviate from a predetermined threshold, a measurement of blood gases is obtained (step 102). The measurement of blood gases may be obtained automatically. The measurement of blood gases may include transcutaneous measurements and/or arterial blood gas measurements. The blood gases may include measurements of PaCO₂. Next, at step 104, if both measurements of expired CO₂ and PaCO₂ measurements are obtained, the gradient between PaCO₂ and EtCO₂ is determined. In some embodiments, the method may further include a step (106) of determining the trend of the gradient. At optional step 108, the method may further include a step of issuing an alert if the measured parameters determined in any one of previous steps deviate from a predetermined threshold. Additional optional step 110 may include automatically initiating/evoking changes in ventilation of the subject so as to return the measured/determined parameters to defined levels.

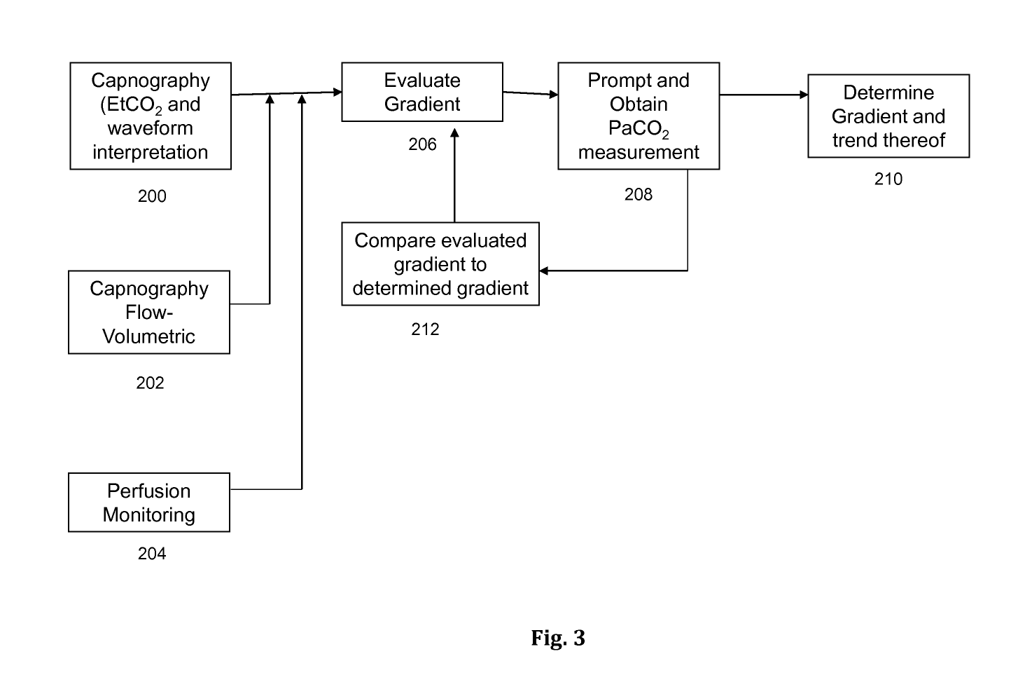

Reference is now made to FIG. 3, which is a schematic block diagram of steps in a method for determining, estimating and/or evaluating the gradient between arterial carbon dioxide partial pressure (PaCO₂) and end tidal carbon dioxide partial pressure (EtCO₂), according to some embodiments. As shown in FIG. 3, measurements of expired CO₂ of a subject are obtained (steps 200-202, obtained by capnography). The measurements may be obtained continuously. Optionally, additional parameters related to the measurements of expired CO₂ are obtained/determined (For example, EtCO₂, CO₂ waveform, dead space, V_{D}/V_{T}, breath flow, volumetric waveform and date derived therefrom, and the like). Routinely, an intermittent measurement of PaCO2 is obtained, at a low frequency (for example, once every 2-4 hours, step 204). The results obtained in previous steps is used by the processing unit to evaluate the PaCO₂ and/or the PaCO₂ to EtCO₂ gradient (step 206). If the obtained measurements of expired CO₂ and/or the parameters related thereto deviate from a predetermined threshold, an immediate (direct or indirect) measurement of blood gases is prompted and obtained (step 208). The measurement of blood gases may be obtained automatically. The measurement of blood gases may include transcutaneous measurements and/or arterial blood gas measurements. The blood gases may include measurements of PaCO₂. Next, at step 210, an accurate determination of the gradient is performed, while optionally also determining the trend of the gradient, so as to provide an accurate assessment as to the condition of the subject. Additional optional step (212) is used to provide a feed-back and enhance learning of the system by comparing between the evaluated PaCO₂ and/or gradient (step 206) and the determined/measured PaCO₂ and/or gradient (step 208). In some embodiments, the method may further include a step of issuing an alert if the measured parameters determined in any one of previous steps deviate from a predetermined threshold and/or a step of automatically initiating/evoking changes in ventilation of the subject so as to return the measured/determined parameters to defined levels.

It is understood by the skilled in the art that the processor of the system is configured to implement the method as essentially described herein.

In the description and claims of the application, each of the words “comprise” “include” and “have”, and forms thereof, are not necessarily limited to members in a list with which the words may be associated.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

### EXAMPLES

### Example 1

### Estimating/Determining the Gradient Between Arterial Carbon Dioxide Partial Pressure (PaCO₂) and End Tidal Carbon Dioxide Partial Pressure (EtCO₂) of a Subject

1) Capnography measurements of EtCO2 and waveform interpretation from a tested subject are obtained. Additionally, volumetric capnography measurements are obtained. The capnography measurements are obtained continuously.
2) Intermittent PaCO2 measurements are obtained at predetermined time intervals and frequency (2-4 hours) from the corresponding perfusion monitoring device (for example, transcutaneous CO₂ measuring device, or arterial blood gas sample).
3) The measurements obtained are used to routinely evaluate the gradient between the EtCO2 and PaCO2,
4) If the measurements in step 1 deviate from a predetermined threshold (for example, changes are observed in EtCO₂ levels, changes are observed in CO₂ waveform, changes are observed in V_{D}/V_{T} as estimated based on stage III of the Volumetric curve), an immediate measurement of PaCO₂ is prompted, and the gradient between PaCO₂ and EtCO₂ is determined. Further determined is the trend of the measured gradient over time.
An exemplary set of data which correlates between changes of V_{D}/V_{T} and the PaCO₂ to EtCO₂ gradient, as determined by the system and method disclosed herein are as follows:

If V_{D}/V_{T} is less than 0.4 then the gradient is about 2 mmHg.

If V_{D}/V_{T} is between 0.4-0.55 then the gradient is about 6 mmHg.

If V_{D}/V_{T} is between 0.55-0.7 then the gradient is about 13 mmHg.

If V_{D}/V_{T} is more than 0.7 then the gradient is about 18 mmHg.

The examples described above are non-limiting examples and are not intended to limit the scope of the disclosure. The described examples may comprise different features, not all of which are required in all embodiments of the disclosure.

What is claimed is:

1. A system for estimating the gradient between arterial carbon dioxide partial pressure (PaCO₂) and end tidal carbon dioxide partial pressure (EtCO₂) of a subject, the system comprising:
a capnograph configured to provide continuous measurements of EtCO₂ and one or more additional breath related parameters of the subject; and
b) a processing unit configured to:
i) provide estimation of PaCO₂ based on the measurements provided by the capnograph; and
ii) provide estimation of the gradient between arterial carbon dioxide partial pressure (PaCO₂) and end tidal carbon dioxide partial pressure (EtCO₂), based on the measurements of EtCO₂ and the estimation of the PaCO₂.

2. The system of claim 1, wherein the breath related parameters comprises: breath flow, respiration rate (RR), respiration effort, CO₂ waveform, V_{D}/V_{T}, volumetric capnography waveform, data derived therefrom, or combinations thereof.

3. The system of claim 1, wherein the processing unit is further configured to provide volumetric capnography, based on the measurements provided by the capnograph.

4. The system of claim 1, wherein the processing unit is further configured to determine the trend of the gradient between the PaCO₂ and the EtCO₂.

5. The system of claim 1, further comprising an alert unit.

6. The system of claim 5, wherein the alert unit is configured to issue an alert if one or more of the values determined by the processing unit deviate from a predetermined threshold.

7. The system of claim 5, wherein the alert comprises visual alert, audible alert, tactile alert, or combinations thereof.

8. The system of claim 1, further comprising a second monitoring device, configured to provide measurements related to PaCO₂.

9. The system of claim 8, wherein the second monitoring device comprises: perfusion monitoring device, transcutaneous CO₂ measuring device, non-invasive PaCO₂ measuring device, Arterial blood gas sampling means, or combinations thereof.

10. The system of claim 8, wherein activation of the second measuring device is initiated in response to deviation of one or more measurements obtained from the capnograph as compared to a predetermined threshold.

11. The system of claim 8, wherein activation of the second measuring device is initiated if one or more of the values determined by the processing unit deviate from a predetermined threshold.

12. A method for estimating the gradient between arterial carbon dioxide partial pressure (PaCO₂) and end tidal carbon dioxide partial pressure (EtCO₂) of a subject, the method comprising:
a. obtaining continuous measurements of EtCO₂ and measurements of one or more additional breath related parameters;
b. estimating PaCO₂ based on the continuous measurements of EtCO2 and the one or more additional breath related parameters;
c. estimating the gradient between the estimated PaCO₂ and measured EtCO₂.

13. The method of claim 12, wherein the breath related parameters comprises: respiration rate (RR), respiration effort, breath flow, volumetric waveform, data derived from the volumetric waveform, CO₂ waveform, data derived therefrom, or combinations thereof.

14. The method of 12, wherein the EtCO₂ measurements are obtained by a capnograph.

15. The method of claim 12 further comprising a step of obtaining a second measurement related to PaCO₂; wherein the second measurement is obtained if the measurements of step a) and/or step b) deviate from a predetermined threshold.

16. The method of claim 15, wherein the second measurement is obtained by a transcutaneous blood gas sensor, arterial blood gas sampling line, non-invasive PaCO₂ measuring device, or combinations thereof.

17. The method of claim 12 further comprising determining the trend of the gradient.

18. The method of claim 17 further comprising issuing an alert if one or more of EtCO₂ and/or the additional breath related parameters and/or the PaCO₂ and/or the gradient and/or the trend of the gradient deviate from a predetermined threshold.
